(19) United States
(12) Patent Application Publication
(10) Pub. No.: US 2024/0090785 A1
(43) Pub. Date: Mar. 21, 2024

(54) Systems and Methods for Detecting Heart Pathologies

(71) Applicant: Apple Inc., Cupertino, CA (US)

(72) Inventors: Narimene Lezzoum, San Jose, CA (US); Michael O’Reilly, San Jose, CA (US); Avery L. Wang, Palo Alto, CA (US); Andrew P. Bright, Los Gatos, CA (US); Ashrith Deshpande, San Jose, CA (US)

(21) Appl. No.: 18/452,422

(22) Filed: Aug. 18, 2023

Related U.S. Application Data
(60) Provisional application No. 63/376,350, filed on Sep. 20, 2022.

(51) Int. Cl. A61B 5/024; A61B 5/00; A61B 7/04; H04R 1/08; H04R 1/22

(52) U.S. Cl. CPC A61B 5/02438; A61B 5/02444; A61B 5/6815; A61B 5/7203; A61B 5/7267; A61B 5/7271; A61B 5/742; A61B 7/04; H04R 1/08; H04R 1/222

(57) ABSTRACT

A device that includes a microphone may be worn in or on an ear of a user. A microphone signal generated by the microphone may be processed to determine a heart activity of a user. An indication of a heart pathology may be detected by applying a predictive algorithm to at least the heart activity. Other aspects are described.

This nonprovisonal patent application claims the benefit of the earlier filing date of U.S. provisional application No. 63/376,350 filed Sep. 20, 2022.

### BACKGROUND

The human heart is an organ that pumps blood through the human body. The heart is the primary organ of the human circulatory system. The human heart includes four main chambers that work in a synchronized manner to circulate blood through the body.

Heart pathologies include a range of conditions that relate to a person's heart, such as, for example, blood vessel disease (e.g., coronary artery disease), heart rhythm problems (e.g., arrhythmias), heart defects (e.g., congenital heart defects), heart valve disease, disease of the heart muscle, heart infection, or other heart pathologies.

### SUMMARY

In one aspect of the disclosure here, a method includes producing a microphone signal with a microphone of a device worn in or on an ear of a user, processing the microphone signal to determine a heart activity, and detecting an association between the heart activity and one or more heart pathologies by applying a predictive algorithm based at least on the heart activity. For example, the method may use the predictive algorithm to detect that the heart activity exhibits characteristics that are known to be associated with one or more heart pathologies. The predictive algorithm may detect signals or characteristics comprised in the heart activity that indicate a risk of one or more heart pathologies.

The method may include capturing a second signal with a second sensor, processing the second signal to determine a second heart activity of the user, and detecting an association between the second heart activity and the one or more heart pathologies by applying the predictive algorithm to the heart activity of the user in the first microphone signal and the second heart activity of the user. The predictive algorithm may detect the heart pathology based on a comparison or correlation of the heart activity of the user and the second heart activity of the user.

The predictive algorithm may include a machine learning model (e.g., neural network) that is trained to detect the heart pathology based on receiving as input the determined heart activity of the user. In some examples, the predictive algorithm detects the heart pathology based on similarity between the heart activity and one or more reference signatures of the heart pathology.

Processing the microphone signal may include applying a filter (e.g., a low pass filter) to the microphone signal. The microphone signals may be processed to sense an infrasound signal in the microphone signal. Additionally, or alternatively, processing the microphone signal to determine the heart activity may include sensing an ultrasound signal in the microphone signal. The microphone signal may be processed to detect a signal that indicates a heartbeat or other movement of such as blood flow that gives information about the heart.

The detected heart pathology may include bradycardia or tachycardia, or other heart pathology. Additionally, or alternatively, the heart pathology may also include an abnormal heartbeat. Other heart pathologies may be detected.

The method may further include determining a seal of the device to the ear of the user to determine a reliability or accuracy of the heart activity of the user. For example, if the seal of the device to the ear is detected as insufficient, the method may put off the processing of the microphone signal or detecting the heart pathology, or both, until the seal is sufficient.

The method may further include determining position (e.g., standing, sitting, lying down, etc.) of the user based on a second sensor, and determining a reliability or accuracy of the heart activity of the user based on the position of the user.

The method may further include presenting an indication of the heart pathology to a display. Additionally, or alternatively, the heart pathology may be output as sound (e.g., as speech through speakers).

The method may further include processing the microphone signal to detect respiratory activity (e.g., a respiratory rate), and detecting the heart pathology further based on the respiratory activity.

In one aspect, a method includes capturing a first heart activity with a first microphone of a first device placed over or in a first ear of a user, capturing a second heart activity with a second microphone of a second device placed over or in a second ear of the user, and detecting an indication of blockage in a carotid artery based on the first heart activity and the second heart activity.

For example, the first heart activity and the second heart activity may be compared. Comparing the first heart activity and the second heart activity may include detecting a difference in strength between the first heart activity and the second heart activity. Additionally, or alternatively, comparing the first heart activity and the second heart activity may include detecting a difference in timing between the first heart activity and the second heart activity.

Detecting the blockage in the carotid artery may include using an artificial neural network to detect the blockage in the carotid artery based on the first heart activity and the second heart activity.

The method may include determining a seal of the device to the ear of the user and determining a reliability or accuracy of the first heart activity of the user. If the seal is determined to be insufficient, the method may turn off processing of the microphone signals of the first microphone until the seal is determined to be sufficient.

The method may include determining position or activity of the user based on a sensor of a device worn by the user and determining a reliability or accuracy of the first heart activity or the second heart activity of the user based on the position or the activity of the user. For example, an inertial measurement unit (IMU) sensor worn on a user may sense a user's position (e.g., whether the user is standing or sitting down), or whether the user is sufficiently still (e.g., stationary, or lacking movement), or both.

In some examples, the method includes indicating the blockage in the carotid artery to the user (e.g., as a message on a display, as a sound, or both).

The first device and the second device may respectively include a first earbud and a second earbud of an earbud pair. Similarly, in some examples, the first device and the second device may have separate housings. In other examples, the first device and the second device may share a common housing (e.g., a single housing of a device worn on a user's head).

In one aspect, a method includes capturing a first heart activity of a user, with a microphone of a hearing device worn by the user, capturing a second heart activity of a user with a sensor of a wearable device worn by the user, and determining a pulse transit time (PTT) based on the first heart activity and the second heart activity of the user. Pulse transit time may refer to the time that a pulse wave takes to travel between two arterial sites (e.g., from the heart to the head).

Determining the PTT may include detecting a difference in timing between the first heart activity and the second heart activity. The sensor may include an electrode that electronically measures the second heart activity of the user.

The method may include synchronizing the microphone and the sensor based on timestamps. For example, the microphone signal of the microphone may be timestamped. Similarly, the sensor signal may be timestamped. Events in the microphone signal may be compared to events in the sensor signal using a common temporal reference.

The method may further include capturing a third heart activity of a user, with a second microphone, and determining the PTT further based on the second heart activity and the third heart activity, where the first microphone is worn in or on a first ear of the user, and the second microphone is worn in or on a second ear of the user.

The PTT, or other findings, may be presented to the user (e.g., on a display or as an audible message). A blood pressure of the user may be determined based on the signal captured inside the ear or the PTT or based at least on the PTT, and similarly presented to the user.

The method may include determining a seal of the hearing device to an ear of the user and determining a reliability or accuracy of the first heart activity of the user based on the seal. For example, if the seal is determined to be insufficient, the first heart activity may be discarded or ignored until the seal is improved.

The method may include determining position of the user based on one or more sensors (e.g., an inertial measurement unit (IMU), a camera, etc.) and determining a reliability or accuracy of the first heart activity of the user or the second heart activity of the user based on the position of the user. For example, if user determined to be standing or laying sideways, the method may ignore or discard the first heart activity, the second heart activity or both, until the position of the user matches a target position (e.g., sitting or lying flat).

In some examples, the sensor includes an electrode or PPG sensor in addition to the acoustic sensor. The sensor may have a separate housing from the hearing device or be worn on a different location of a user's body than the hearing device, or both. For example, the sensor may be worn on a user's wrist or on a user's chest (e.g., near the heart).

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have advantages not specifically recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 shows a system for using an ear-worn device to detect heart pathology, in accordance with some aspects.

FIG. 2 shows an example of a flow diagram for processing a microphone signal to predict a heart pathology, in accordance with some embodiments.

FIG. 3 shows an example system for detecting blockage of a carotid artery, in accordance with some aspects.

FIG. 4 shows an example of a system for measuring a pulse transit time using an ear-worn microphone, in accordance with some aspects.

FIG. 5 illustrates an example of a method for determining a heart pathology based at least on heart activity sensed with a microphone, in accordance with some aspects.

FIG. 6 illustrates an example of a method for detecting a potential blockage of the carotid artery, based at least on heart activity sensed with a microphone, in accordance with some aspects.

FIG. 7 illustrates an example of a method for determining a pulse transit time (PTT), based at least on heart activity sensed with a microphone, in accordance with some aspects.

FIG. 8 illustrates an example of an audio processing system, in accordance with some aspects.

### DETAILED DESCRIPTION

A device may include one or more microphones or other sensors that sense heart activity of the user. The heart activity is extracted from a signal (e.g., a microphone signal or other sensor signal).

Heart activity may include heart movement such as contraction of the left or right atrium and ventricle, and movement of blood through the heart. The heart activity may include the cardiac cycle of the heart (e.g., a heartbeat), which indicates the phases of heart relaxation (diastole) and contraction (systole). Under normal heart activity, the ventricular diastole begins with isovolumic relaxation, then proceeds through three sub-stages of inflow, namely: rapid inflow, diastasis, and atrial systole. The heart activity may indicate an underlying heart pathology or risk of a heart pathology.

A heart pathology may include a disease or abnormality of the heart that may result in a reduced ability of the heart to effectively pump blood through the human body. Such heart pathology may be identified by or associated with irregular heart activity.

Earphones, headphones, and other hearing devices may be used for listening to music, noise cancellation and/or hearing enhancement. In some aspects of the present disclosure, these devices may be equipped with acoustic transducers (e.g., microphones) that are arranged to capture sounds inside the ear (e.g., in a user's ear canal). In some examples, the same or different microphones may be used for active noise cancellation, transparency, and adaptive equalization functions that are implemented in the hearing device. Acoustic transducers may sense sound (e.g., vibrations) and generate a signal (e.g., a microphone signal) that varies in magnitude over time and/or frequency.

The role of earphones, headphones, or other hearing devices may be expanded to support the creation of a phonocardiogram and ballistocardiograph.

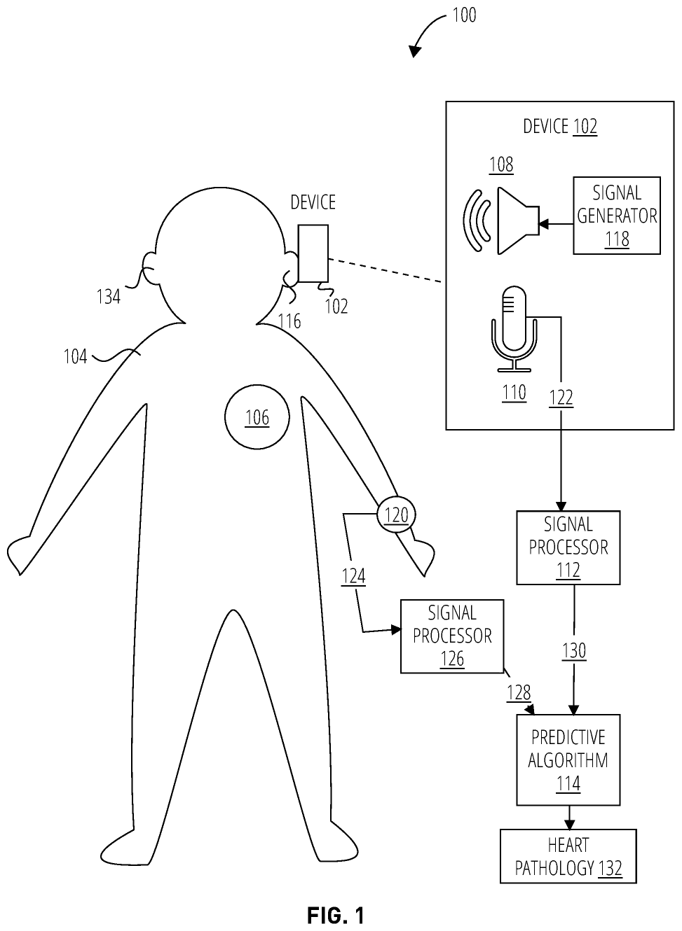

FIG. 1 shows a system 100 for using an ear-worn device to detect heart pathology, in accordance with some aspects. Some or all of blocks described in this example or in other examples may be performed by processing logic. Processing logic may comprise hardware (e.g., circuitry, dedicated logic, programmable logic, a processor, a processing device, a central processing unit (CPU), a system-on-chip (SoC), machine-readable memory, etc.), software (e.g., machine-readable instructions stored or executed by processing logic), or a combination thereof.

A device 102 may be worn on or in an ear 116 of a user 104. The device 102 may include in-ear sensing technologies (e.g., microphone 110 and IMUs), and apply one or more algorithms (e.g., a machine learning model) to detect heart, vascular, pulmonary, and neurologic related pathology. The algorithms may assess the severity of the pathology and predict the appropriate therapies. In some aspects, data from various sensors (e.g., from other wearable devices or from mobile devices) and/or data from other sources of data (e.g., patient records stored digitally) may be processed and considered as a whole to detect heart pathologies. Consent from a user may be obtained before collection of personal data or biometric information. Correlations between the various sensed signals may be learned and leveraged to detect pathologies. For example, based on a sufficiently large dataset, a correlation of a sensor A that senses heart activity ‘X’ to a sensor B that senses heart activity ‘Y’ may indicate a risk for pathology ‘Z.’ A predictive algorithm detects a pathology ‘Z’ in a user when sensor A exhibits heart activity ‘X’ and sensor B exhibits heart activity ‘Y’. Detecting a pathology may include detecting an elevated risk of the pathology.

Device 102 may include a headphone (e.g., an earbud) that is worn on or in an ear 116 of a user 104. The device 102 may include a microphone 110 that generates a microphone signal 122. At a block referred to as signal processor 112, processing logic may process the microphone signal 122 to determine a heart activity 130 of a user 104 in the microphone signal 122.

At a block referred to as a predictive algorithm 114, processing logic may detect heart pathology 132 by applying the predictive algorithm 114 based at least on the heart activity 130. As described, the heart activity 130 may include heart movement of heart 106 such as contraction of the left or right atrium and ventricle, or movement of blood. Heart activity 130 may include the expansion and contraction of arteries throughout the body, for example, arteries that are located at or around the user's ears. Heart activity may include a waveform that varies in magnitude over time and/or frequency to correspond to movement of the heart or blood.

In some examples, a second signal 124 is captured with an optional second sensor 120. The second sensor 120 may include an accelerometer, an electrode, a second microphone, or other sensor or combination of such sensor. In some examples, the second microphone may be worn in a second ear 134 of the user. The second signal 124 may be processed by the signal processor 112 to determine a second heart activity 128 of the user. Although the heart activity 130 and the second heart activity 128 may originate from the same user and the same heart 106, the second heart activity 128 may be different from the heart activity 130 (obtained through the microphone signal 122) due to differences in location of the sensed heart activity and/or due to the nature of the sensors.

The processing logic may predict the heart pathology 132 by applying the predictive algorithm 114 to both the heart activity 130 of the user and the second heart activity 128. For example, the predictive algorithm 114 may compare the two heart activities or associate behavior of the two heart activities to a given heart pathology 132.

In some examples, the second sensor 120 may have a separate housing from device 102. The separate housing may be worn or coupled to the user 104 at a different location of the user than device 102.

The predictive algorithm 114 may include an artificial neural network or other machine learning model that is trained to detect the heart pathology based on the heart activity of the user. For example, an artificial neural network may be trained with a sufficiently large dataset (e.g., training data) of heart activity signals with various corresponding heart pathologies to reinforce the artificial neural network to associate a given heart activity with a particular heart pathology. The training data may include ground truth data that includes real measurements of heart activity which may be labeled (e.g., ‘normal,’ ‘aortic murmur,’ ‘abnormal,’ etc.). In some aspects, the predictive algorithm 114 may include a plurality of machine learning models, as described in other sections.

The predictive algorithm 114 may detect the heart pathology 132 based on similarity between the heart activity 130 and one or more reference signatures of the heart pathology. For example, the predictive algorithm 114 may refer to a database of reference signatures (e.g., signals) that are each associated with a heart pathology (e.g., aortic murmur, bradycardia, tachycardia, aortic stenosis, mitral regurgitation, aortic regurgitation, mitral stenosis, patent ductus arteriosus, etc.). Each signature may include one or more cardiac cycles which may be compared against the heart activity 130. The predictive algorithm 114 may determine the heart pathology 132 in response to the heart activity 130 being within a threshold similarity to one or more reference signatures corresponding to the heart pathology 132. Processing logic may refer to reference data such as stethoscope, duplex doppler, ultrasound, Echocardiogram, medical diagnosis, or other data or a combination thereof, to correlate the sensed heart activity 130 or the second heart activity 128 to a heart pathology 132.

In some examples, processing the microphone signal 122 at the block referred to as the signal processor 112 includes applying a filter (e.g., a high pass filter, a low pass filter, or both) to the microphone signal. Processing logic may sense the full bandwidth signal or an infrasound signal (e.g., at a frequency below the human audible frequency range) in the microphone signal. Additionally, or alternatively, processing the microphone signal 122 to determine the heart activity 130 may include sensing an ultrasound signal in the microphone signal. A signal generator 118 may provide an acoustic signal (full bandwidth, ultrasound signal, an infrasound signal, or a combination thereof), to drive a speaker 108 that is worn on or in device 102. The speaker 108 outputs the respective signal into the ear 116 of user 104. The microphone 110 senses this pilot signal (e.g., the ultrasound and/or infrasound signal) as it reflects off the various surfaces of the interior of ear 116, and the signal processor 112 extracts the heart activity 130 based on variations in the sensed pilot signal.

In some examples, processing logic may determine a seal of the device 102 to the ear 116 of the user to determine a reliability or accuracy of the heart activity 130 of the user. For example, processing logic may sense a loss in the pilot signal exceeds a threshold amount. In another example, processing logic may detect a threshold amount of ambient noise carried in microphone signal 122. Ambient noise may be understood as sounds around the user outside of the enclosed space between the device and the user's ear canal (e.g., an air conditioner, a television, car traffic, etc.). If the loss of the pilot signal or the amount of ambient noise satisfies a threshold, processing logic may presume that the seal is insufficient and ignore the heart activity sensed by the microphone 110. The predictive algorithm 114 may be halted and resumed when the seal is deemed to be sufficient (e.g., the threshold is no longer satisfied). A sufficient seal may be formed when an earbud is properly placed in a user's ear canal and gaps between the earbud and ear canal are sufficient to reduce loss of the reflected pilot signal and reduce pickup of ambient noise. In some examples, before running the operations to detect the heart pathology 132, in the device 102, processing logic may perform the seal measurement test to ensure the captured heart activity is of sufficient quality. After the seal measurement test passes, the signal processing and prediction algorithms to detect the heart pathology 132 may be activated.

In some examples, processing logic may determine to capture the signal at a different tap point in the DSP chain. For example, in the ANC or transparency chains to extract a high fidelity signal.

In some examples, processing logic may determine a position of the user based on a second sensor and determine a reliability or accuracy of the heart activity of the user based on the position of the user. For example, the second sensor 120 may include an inertial measurement unit (IMU) that can be processed at signal processor 126 to track the user's position and determine whether the user is walking, standing, or sitting. Processing logic may halt predictive algorithm 114 based on the sensed position of the user, for example, processing logic may halt the predictive algorithm 114 and ignore the heart activity 130 until the user is sensed to be sitting.

In some examples, processing logic may present an indication of the heart pathology to a display. For example, device 102 or another device (e.g., a mobile phone, a smart watch, a head up display, a computer, etc.) may include a display that presents the heart pathology or lack of heart pathology (e.g., ‘normal heart activity’) to the user. Additionally, or alternatively, the heart pathology may be stored digitally in computer readable memory. In some examples, the heart pathology and heart activity may be securely stored and timestamped. For instance, this data may be securely stored and associated with user 104 on device 102 or another device that has been associated with user 104. The data may be available for future reference. In some examples, the data may further be tagged with the position or activity of the user (e.g., sitting, laying down, standing, walking, running, etc.) so that processing logic may analyze the data with respect to the user's position or activity.

In some examples, processing logic may detect respiratory activity of user 104, and detect the heart pathology 132 further based on the respiratory activity. For example, the microphone signal 122 or the second signal 124 may be processed to determine a respiratory rate of user 104. The predictive algorithm 114 may determine the heart pathology 132 based on both the heart activity and the respiratory rate. For example, the speed of the respiratory rate may be considered in view of the user's heart rate to determine if the heart rate is normal, fast, or slow. In some examples, processing logic may refer to a database of heart activity that corresponds to respiratory rates, which may be labeled as normal or having a heart pathology. In other examples, processing logic may apply a machine learning model (e.g., an artificial neural network) that is trained to detect a heart pathology with the heart activity and the respiratory activity as input.

As such, each sensor (the microphone and/or additional sensor(s)) can detect the activity and timing of each heartbeat. Processing logic can implement algorithms to detect rate and rhythm abnormalities, for example, bradycardia and tachycardia. Processing logic may detect abnormal heart rhythms and indicate atrial fibrillation by identifying an irregular rhythm. Further, processing logic may measure other rhythm abnormalities by combining a ballistic signal with one or all of these signals: voice accelerometer signal, microphone or IMU to determine whether an abnormal beat is originating from the ventricle or the atrium of the user's heart. Because processing logic can detect the timing of every heartbeat, the various permutations of heart rate variability and heart rate entropy may be determined. The heart activity may include a phonocardiogram that indicates and identifies specific valvular abnormalities. Using machine learning and combining the ballistic signal, processing logic may predict the results of ground truth assessments of valve function. In some aspects, processing logic may predict the valve area and gradient in users with aortic stenosis.

In some aspects, processing logic may determine a cardiac pump function by predicting the ejection fraction of heart 106. By combining an acoustic signature from the user's lung with the heart activity 130 obtained from the microphone signal 122, processing logic may determine the severity of congestive heart failure and the response to treatment by user 104.

Processing logic may concomitantly measure activity through the various sensors (e.g., the microphone 110 and the second sensor 120) to capture signals at rest and after activity. Specific cardiac indices may be generated and referenced that relate to both cardiac function and valvular abnormalities of the human heart. As discussed, a motion sensor may be used to determine the position of the user at which these sounds have been detected which may indicate different pathologies depending on the position of the person and the detected heart activity.

In addition to valvular abnormalities and cardiac function, processing logic may detect carotid artery stenosis by sensing the characteristic bruit (or sound) of carotid artery stenosis. Heart pathologies such as vascular abnormalities (e.g., cerebral artery or other aneurysms) may also be detected. By sensing and processing the respiratory activity with the cardiac activity, processing logic may assess heart pathology with a robust assessment of cardiopulmonary coupling.

The detection for heart pathology 132 may be performed by sensing the heart activity 130 and the second heart activity 128 simultaneously in view of the combined signals. Processing logic may identify heart murmurs or extra heart sound sensed by the microphone 110.

The device 102 (e.g., a hearing device, a headphone, an earbud, or other ear-worn device) of this example or of other examples may output audio content such as music, a podcast, a news broadcast, audio of a movie or other audiovisual work, or other audio content. Processing logic may output an inaudible pilot signal into the user's ear canal and sense the pilot signal with a microphone of the device, as discussed. The pilot signal may be output concurrently with the audio content, or not. The reflections of the pilot signal may be sensed in the microphone of the device by filtering out the output audio content or other noises. In such a manner, the device may be used as a hearing device for outputting content while also measuring a user's heart activity in the numerous examples discussed. Processing logic may measure the user's heart activity repeatedly over a period of time to obtain a deep understanding of the user's heart activity.

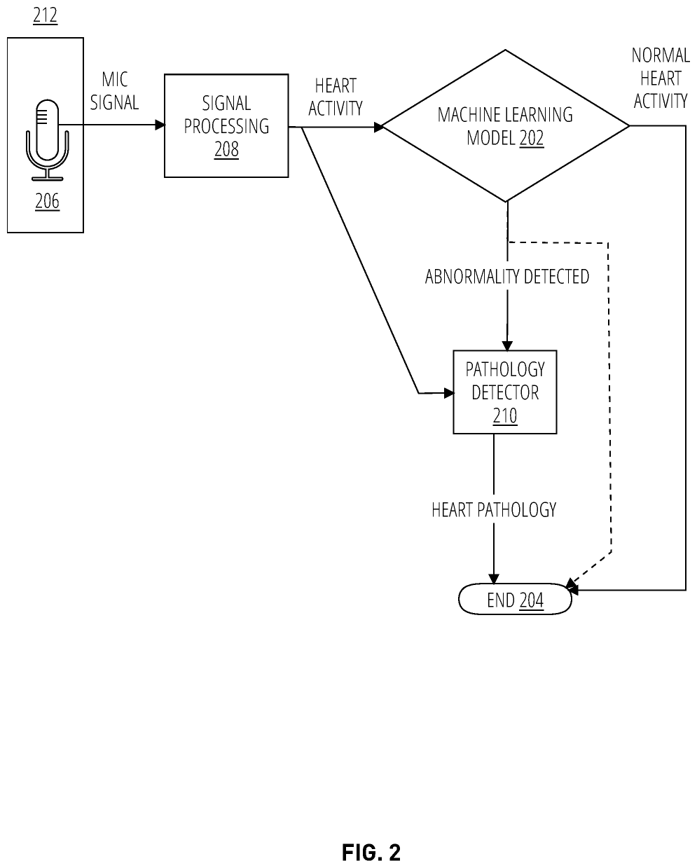

FIG. 2 shows an example of a flow diagram for processing a microphone signal to predict a heart pathology, in accordance with some embodiments.

Microphone 206 may be housed in a device 212 that is worn in or on an ear of a user. The device 212 may include a headphone, earbud, or a head-mounted display (HMD). Some or all the blocks may be performed by processing logic. The signal processing block 208 may apply to numerous examples (e.g., to the signal processing blocks described in other sections). Similarly, a block referred to as machine learning model 202 and a block 210 labeled pathology detector may apply to numerous examples (e.g., to the prediction algorithm blocks described in other sections).

At signal processing block 208, processing logic may obtain the raw microphone signal from microphone 206 and extract heart activity. This may include sensing a pilot signal (e.g., an ultrasound signal) as it reflects off the user's ear canal. The pilot signal may vary in intensity over time and frequency as it is output by speakers into the user's ear, as discussed. Variations in the sensed pilot signal (as picked up by microphone 206) may be sensed at signal processing block 208 to correspond to movement of the wearer's heart. Processing logic may generate heart activity that corresponds to this sensed movement of the user's heart or blood coursing through the user's circulatory system. For example, for example, processing logic may filter or shape the microphone signal or construct a new signal based on the sensed movement, or a combination thereof. The heart activity may include heart movement (diastole and systole) over one or more cardiac cycles.

The heart activity may be provided as input to machine learning model 202. Machine learning model 202 may include an artificial neural network trained to detect whether the input heart activity is normal or abnormal. In response to the machine learning model producing an output indicating normal activity, processing logic may proceed to block 204. The finding of a normal heart activity may be electronically stored, presented to a user, or both.

If machine learning model 202 produces an output indicating abnormal heart activity, processing logic may proceed to a block 210 labeled pathology detector to further detect one or more heart pathologies associated with the heart activity in more detail. In other embodiments, processing logic may simply proceed to block 204 (rather than proceed to block 210) and present or save the finding that an abnormal heart activity was sensed of the user. Any health-related findings (e.g., heart pathology, PTT, etc.) and biometric information may be securely stored within a device, for example, as private to the user and requiring user authentication for access.

At block 210, processing logic may apply a second machine learning model (e.g., a second artificial neural network) to the heart activity. The second artificial neural network may be trained to receive the heart activity as input and to output a heart pathology that corresponds to the heart activity. Processing logic may proceed to block 204 and store the detected heart pathology in computer readable memory, display the heart pathology, or both. The data (e.g., a finding) may be timestamped.

Training an artificial neural network (to detect abnormality and/or heart pathology) can involve using an optimization algorithm to find a set of weights to best map inputs (e.g., heart activity, and/or features extracted from the heart activity) to outputs (e.g., a finding of a heart pathology or lack thereof).

These weights may be values that represent the strength of a connection between neural network nodes of the artificial neural network. During training, the machine learning model weights can be trained to minimize the difference between a) the output (e.g., whether an abnormality is detected, or an output heart pathology) generated by the machine learning model based on the input training data, and b) approved output (e.g., whether an abnormality is detected, or which heart pathology was detected) that is associated with the training data. The heart activity and approved output of the training data can be described as input-output pairs, and these pairs can be used to train a machine learning model in a process that may be referred to as supervised training.

The training of the machine learning model can include using non-linear regression (e.g., least squares) to optimize a cost function to reduce error of the output of the machine learning model (as compared to the approved findings of the training data). Errors (e.g., between the heart pathology output and the approved pathology output) are propagated back through the machine learning model, causing an adjustment of the weights which control the neural network algorithm. This process may be performed repeatedly for each recording, to adjust the weights such that the errors are reduced, and accuracy is improved. The same set of training data can be processed a plurality of times to refine the weights. The training can be completed once the errors are reduced to satisfy a threshold, which can be determined through routine test and experimentation.

In some examples, the machine learning model 202 or an ML model at block 210 may take multiple signals sensing heart activity from two or more locations. For example, either ML model may receive the heart activity sensed by microphone 206 and a second heart activity sensed by a photoplethysmography (PPG), accelerometer, EKG, etc. The ML model may be trained to output whether an abnormality is detected, or to classify the sensed heart activities, based on the multiple signal inputs.

Other pathology detection algorithms may be implemented that do not rely on machine learning. For example, in the machine learning model 202, a similarity-based algorithm may be applied to the heart activity. The similarity-based algorithm may compare the heart activity to one or more reference normal activity signatures. For example, the algorithm may detect whether variations of the heart activity signal over time and/or frequency are similar to the reference signatures within a threshold margin. If not, then processing logic may proceed to block 210. If so, then processing logic may deem the heart activity as normal and proceed to block 204. Similarly, at block 210, a similarity-based algorithm may be applied to the heart activity that detects whether variations in the heart activity are similar over time and/or frequency to various stored reference heart activity signatures that may each correspond to a heart pathology. If the similarity between the sensed heart activity and one or more of the reference heart activity signatures is within a threshold similarity, then processing logic may determine the heart pathology as that which corresponds to the one or more reference heart activity signatures. Processing logic may proceed to block 204.

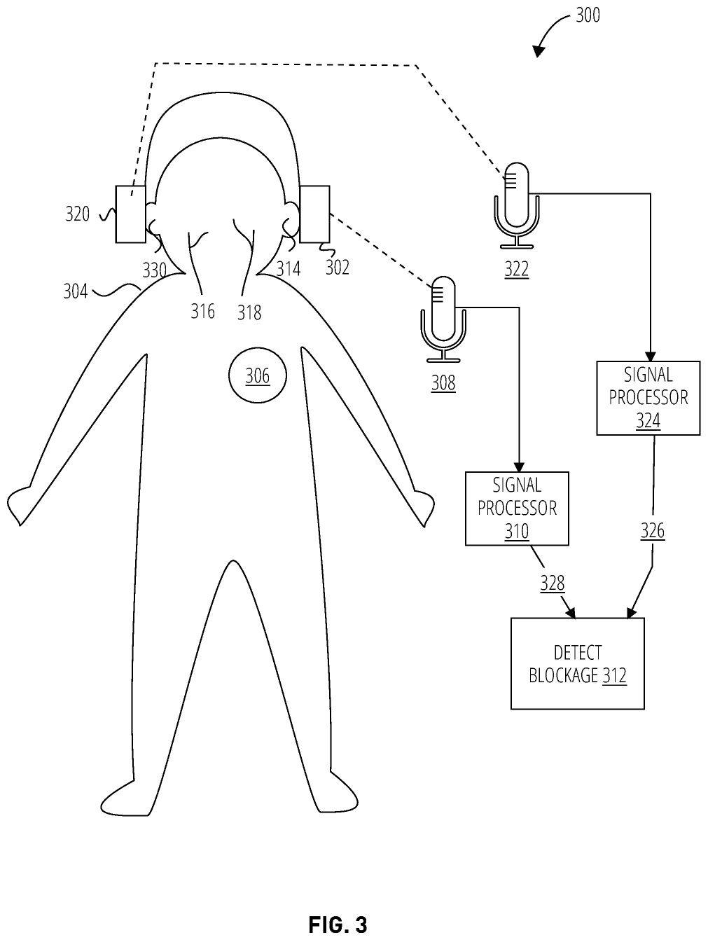

FIG. 3 shows an example system for detecting blockage of a carotid artery, in accordance with some aspects.

Carotid artery stenosis (narrowing of the blood vessels that make up the carotid artery) typically goes undetected until a person has a brain stroke. The narrowing often results from atherosclerosis, a build-up of plaque on the inside of the arteries which may be referred to herein as blockage. Diagnosis of such condition is traditionally performed at a hospital using dedicated equipment (e.g., a duplex Doppler system).

The carotid artery may include a left carotid artery 318 and a right carotid artery 316. The left carotid artery 318 may include a left common carotid artery and an internal and external carotid artery that branches from the left common carotid artery. Similarly, the right carotid artery may include a right common carotid artery and an internal and external carotid artery that branches from the right common carotid artery. The right common carotid artery extends up the neck from the innominate artery which is a major branch off the aorta. The left common carotid artery branches directly off the aorta. The left and right carotid arteries carry blood and oxygen to the brain, head, and face. A clot or blockage in either of the carotid arteries can give rise to serious health issues such as ischemic transient attack (e.g., brain stroke).

A system 300 may detect the presence or absence of heart activity inside either ear canal of a user. Signal processing techniques may be implemented to determine the loudness or strength of these signals. Temporal differences between reception or non-reception of these signals at each ear may be analyzed to determine whether or not a user has blockage in the carotid artery (e.g., the left carotid artery or the right carotid artery).

In one aspect, system 300 includes a first device 302 and a second device 320. The first device 302 may include a microphone 308 that is arranged to sense sound in the interior of a first ear 314. Similarly, the second device 320 may include a second microphone 322 that is arranged to sense sound in the interior of a second ear 330.

In some examples, the first device 302 and the second device 320 are respectively a first earbud and a second earbud of an earbud pair. Earbuds may be worn at least partially inside a user's ear canal. A portion of the earbud may plug into the ear canal thereby forming a seal with minimal or no openings between the earbud and the user's ear. Each of the microphones may be positioned to capture sounds inside of the user's ear canal. Further, each of the earbuds may have speakers to output sound into the user's ear canal. In other examples, each of the first device 302 and the second device 320 may comprise a headphone that is worn over the user's ear. Each headphone may include an ear cup and a cushion that is worn over the user's ear to form a seal. The microphone may be arranged in the ear cup to pick up sounds within the user's ear. In some examples, such as with earbuds or with some headphones, each of the first device 302 and the second device 320 may have separate housings.

System 300 may include processing logic that performs operations (e.g., at blocks 310, 312, 324, or other operations) to detect whether a blockage of the carotid artery is present. Processing logic may process signals (e.g., microphone signals) that capture sound at each ear to detect if a carotid artery has any blood blockage. Processing logic may obtain the signals at both ears and analyze relationships or correlations between the audio signals.

A first heart activity 328 is captured with the microphone 308 of a first device placed over or in the first ear 314 of a user. A second heart activity 326 is captured with the second microphone 322 of a second device 320 placed over or in the second ear 330 of the user. As described, heart activity may refer to movement of the heart 306 (e.g., diastole and systole) or movement of blood through the user's circulatory system which indicates such heart movement. The first heart activity and second heart activity may measure the activity of the same heart 306, but at distinct locations (e.g., at opposite ears of the user).

Each of the first heart activity 328 and the second heart activity 326 may be determined by processing the respective microphone signals (at block 310 and block 324) as described in other sections. For example, each microphone signal may be filtered to sense variations in a sensed ultrasound or infrasound pilot signal, to determine the respective heart activity.

Processing logic may, at block 312, detect blockage in a carotid artery (e.g., in the left carotid artery 318 or the right carotid artery 316) based on the first heart activity 328 and the second heart activity 326. For example, at block 312, processing logic may compare the heart activities or analyze the first heart activity 328 and the second heart activity 326 in view of each other to detect a blockage in the carotid artery.

Processing logic may detect a difference between the first heart activity and the second heart activity and if the difference satisfies a given threshold, processing logic may determine that the carotid artery has a blockage.

In some examples, processing logic may detect a difference in strength between the first heart activity and the second heart activity and if the difference satisfies a given threshold, processing logic may determine that the carotid artery has a blockage. The location at which the heart activity is measured to be weaker may indicate the location of the blockage. For example, if the first heart activity (measured at the left ear) is stronger than the second heart activity (measured at the right ear) by greater than the threshold amount, processing logic may determine that the right carotid artery is blocked. Strength may include a measure of signal amplitude.

Additionally, or alternatively, processing logic may detect a difference in timing between the first heart activity and the second heart activity. If the difference satisfies a threshold, then processing logic may determine that the carotid artery has a blockage. The location at which the heart activity is delayed may indicate the location of the blockage. For example, if the first heart activity (measured at the left ear) is sensed to occur before the second heart activity (measured at the right ear) by more than the threshold amount, processing logic may determine that the right carotid artery has blockage.

In some examples, detecting the blockage in the carotid artery includes applying an artificial neural network to detect the blockage in the carotid artery based on the first heart activity 328 and the second heart activity 326 as inputs. For example, an artificial neural network may be trained with training data that includes heart activity measured acoustically from each ear, accompanied with a targeted output such as a blocked carotid artery, blocked left carotid artery, blocked right carotid artery, etc. Training of an artificial neural network may be performed through techniques such as those described in other sections, or other known techniques not described.

As described with respect to other examples, processing logic may perform a seal test at the first device 302 or at the second device 320, or both, to determine reliability or accuracy of the respective signals. Processing logic may perform the operations to detect the blockage in response to when the seal between the device (e.g., an ear bud or headphone) is sufficient. Otherwise, processing logic may halt performance of blockage detection until the seal is sufficient and both heart activities 328 and 326 are deemed to be dependable.

Further, processing logic may determine a position of the user 304 based on a sensor of a device worn by the user. This sensor (e.g., an accelerometer, a gyroscope, an IMU, a camera, etc.) may be integral to the second device 320, the first device 302, or another device (not shown). Processing logic may determine a reliability or accuracy of the first heart activity 328 or the second heart activity 326, or both, based on the sensed position or activity of the user. For example, if the user is standing, walking, or not sitting, processing logic may deem the first heart activity 328 or the second heart activity 326 to be unreliable and halt some or all the blockage detection operations. This may improve efficiency of the operation and preserve CPU and energy resources.

In some examples, processing logic may present an indication of the blockage in the carotid artery to a display such as to a mobile phone, a tablet computer, a computer display, a wristwatch, an HMD, or other display. The indication may include whether or not a blockage is detected in the carotid artery or where the blockage is detected, such as in the left carotid artery or the right carotid artery.

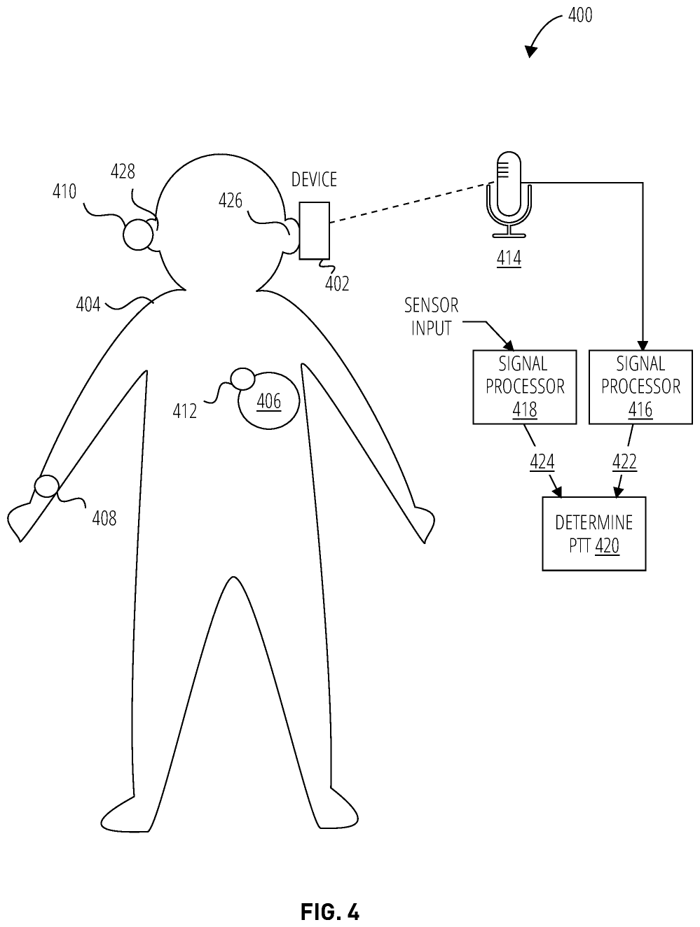

FIG. 4 shows an example of a system 400 for measuring a heart-related metric using an ear-worn microphone, in accordance with some aspects. The system 400 may utilize a set of wearable devices at distinct locations of the body to sense heart-related metrics such as pulse transit time (PTT). These wearables may include different sensors such as microphones, electrodes, or PPGs, which are used to estimate the PTT between the two locations. Estimating the PTT may provide health indicators for the heart and arteries.

PTT refers to the time it takes a pulse wave to travel between two arterial sites, such as from the aortic valve to another location on the human body. The speed at which this arterial pressure wave travels may be proportional to blood pressure.

A system 400 may include a hearing device 402 (e.g., an ear-worn device) that includes a microphone 414. The system 400 may include at least one sensor other than microphone 414, such as sensor 408, 412, or 410, that is worn at a different location of the user than device 402. The sensor may be integral to a wearable device (e.g., a watch or chest-strapped device) or a mobile device (e.g., a phone or tablet computer).

The system may include processing logic that performs various operations to determine a PTT. Processing logic may obtain or capture a first heart activity 422 of user 404, with the microphone 414 of hearing device 402 worn by the user 404. As described in other sections, processing logic may obtain the microphone signal of microphone 414 and extract the first heart activity 422 from the microphone signal at signal processor block 416. Although not shown in this example, device 402 may include a speaker that outputs a pilot signal (e.g., an infrasound or ultrasound signal). The microphone 414 senses the pilot signal as it travels and reflects off surfaces within the user's ear. The first heart activity 422 may be generated to correspond to variations of the sensed pilot signal, as described in other sections.

Processing logic may capture a second heart activity 424 of a user with a sensor (e.g., sensor 410, 408, or 412) of a wearable device worn by the user. In some examples, any of the sensors may be housed on or in a worn device such as a wrist-worn device, a chest-worn device, an earbud, a finger worn device, other ear-worn device (or any other body worn device that may give information about the blood flow through the human body). Additionally, or alternatively, any of the sensors may be housed on or within a mobile device that is placed on or against the user (e.g., a mobile phone or other device).

Processing logic may determine the PTT at block 420 based on the first heart activity 422 and the second heart activity 424 of the user. Determining the PTT may include detecting a difference in timing between the first heart activity 422 and the second heart activity 424. For example, processing logic may determine that the first heart activity occurred at time T and a second heart activity which may represent the same heart activity but at a different location, occurs at time T−x. Processing logic may determine the PTT as being the difference x, which may be offset based on other considerations.

In some examples, any of the sensors (e.g., 410, 408, or 412) may include an electrode (serving as an electrocardiogram) that electronically measures the second heart activity 424 of the user. For example, a user may place her finger on the electrode to obtain an electronically measured activity of the heart 406. Given that the electronic signal from the heart travels at a known and much faster speed compared to the pulse wave from the heart 406 to the user's ear 426, the lag from the time the second heart activity 424 is measured to the first heart activity 422 may indicate the travel time (e.g., PTT) of the pulse wave from the heart 406 to the user's ear 426.

Additionally, or alternatively, the sensors may include a PPG sensor. A PPG sensor may include a light emitter and photodetector that senses volumetric variations of blood circulation (e.g., in a capillary vessel). In some examples, the PPG may be placed on the user's chest (e.g., at or near the user's heart 406). The PTT may be determined based on the time difference between the second heart activity 424 (as sensed by sensor 412) and the first heart activity 422.

At block 418, processing logic may extract the second heart activity from the sensor signal. The signal processing may differ depending on the sensor technique. For example, the signal processing may identify a heartbeat in an EKG signal or a PPG signal.

The microphone 414 and any of the sensors 408, 410, and 412 may be synchronized. For example, the microphone 414 or each of the sensors may operate on its own clock or a clock of the device in which it is housed. The signals from the microphone and each sensor may be timestamped based on its respective device clock. The timestamps of the microphone signal and each of the sensor signals may be synchronized using a timestamp synchronization technique. For example, processing logic may refer to a common clock and an offset to be applied to each of the signals with reference to the common clock. Various timestamp synchronization techniques may be implemented.

In some examples, in addition to a second sensor (e.g., an EKG or PPG) and second heart activity 424, a third heart activity of a user is captured with a second microphone (e.g., sensor 410) which is worn in or on a second ear of the user 404. Processing logic may determine the PTT further based on the second heart activity 424 and the third heart activity. For example, hearing device 402 may be worn on or in ear 426. The PTT may be measured based on the lag between the heart activity sensed by EKG or PPG of sensor 408 or 412 and that sensed by microphone 414. Concomitantly, another PTT measurement may be made based on the lag between heart activity sensed by EKG or PPG sensor of sensor 408 or 412, and that sense by a microphone of sensor 410 worn at the opposite ear 428 of the user 404. As such, processing logic may consider both PTT measurements from the heart to the ear to determine the PTT (e.g., as an average or other combination of the two measurements).

The PTT may be securely stored (e.g., as an electronic record) in computer readable memory, which, as an example, may be local to a device. The PTT may be presented acoustically through speakers or visually to a display of any of the devices, or of a different device.

In some examples, processing logic may determine a blood pressure of the user based at least on the PTT. For example, the PTT and blood pressure may have an inverse relationship. As such, processing logic may determine the BP as an inverse of the PTT. In some examples, processing logic may account for other factors such as a user's age, etc., and apply an offset or other correction. As such, processing logic may estimate whether a user has low blood pressure, high blood pressure, of other heart pathologies such as a heart, arterial, or valvular based pathology.

As described with respect to other examples, processing logic may test the seal of the hearing device 402 to the user's ear 426 and determine a reliability or accuracy of the first heart activity 422 of the user based on the seal. Similarly, processing logic may determine a position or activity of the user 404 based on a sensor (e.g., sensor 408, 410, 412, or another sensor), which may include an accelerometer, gyroscope, IMU, camera, or other sensor or combination thereof. Processing logic may determine reliability or accuracy of any of the heart activities based on the position or activity of the user as described in other sections, and halt determination of the PTT until the position or activity of the user is at a desired position or activity level (e.g., not running or walking).

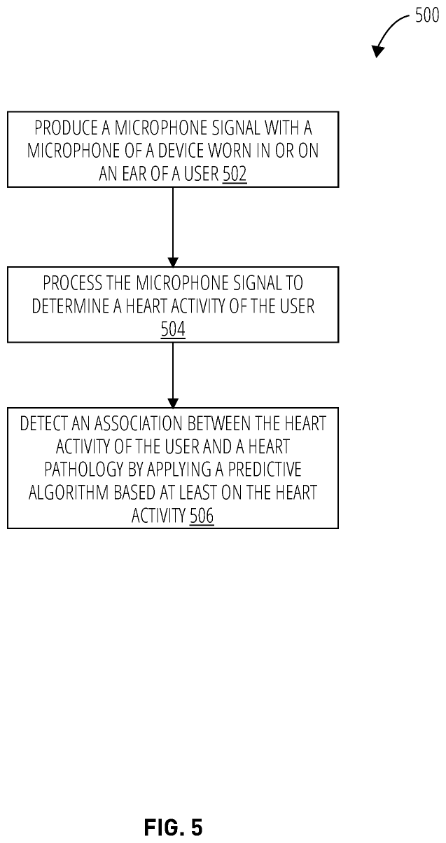

FIG. 5 illustrates an example of a method 500 for determining a heart pathology based at least on heart activity sensed by a microphone, in accordance with some aspects. The method may be performed with various aspects described. The method may be performed by processing logic of an audio processing device. Processing logic may include hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof.

Although specific function blocks (blocks”) are described in this and other examples, aspects are well suited to performing various other blocks or variations of the blocks recited in the method. It is appreciated that the blocks may be performed in an order different than presented, and that not all of the blocks may be performed.

At block 502, processing logic captures a microphone signal with a microphone of a device worn in or on an ear of a user. The device may include a hearing device that includes a speaker, as described in other sections. In some examples, the device includes an earbud that is worn in the ear of the user. When worn properly, the earbud housing may create a seal with the user's ear canal (e.g., the external auditory meatus) which may improve acoustic sensing of heart activity.

At block 504, processing logic processes the microphone signal to determine a heart activity of a user in the microphone signal. As described, the device may include a speaker that is arranged to output an acoustic signal to the user's ear canal. Reflections of the signal are captured in the microphone signal and variations of this sensed signal may be used to reconstruct or determine the heart activity. In some examples, prior to determining the heart activity of the user via the microphone signal, processing logic may test the seal of the device against the user's ear, as described in other sections.

At block 506, processing logic detects an association between the heart activity and a heart pathology by applying a predictive algorithm based at least on the heart activity. The heart pathology may include an aortic murmur, bradycardia, tachycardia, aortic stenosis, mitral regurgitation, aortic regurgitation, mitral stenosis, patent ductus arteriosus, or other heart pathology. The heart pathology may include an abnormal heart activity, heart rhythm, or heartbeat, such as heart activity that deviates from a normal or healthy heart activity in one or more cardiac cycles. The association may link the heart activity to risk of a given heart pathology. For example, a heart pathology with such a characteristic waveform may have a higher probability or risk towards one or more heart pathologies, based on ground truth data.

The predictive algorithm may include one or more machine learning algorithms (e.g., an artificial neural network) as described. Additionally, or alternatively, the predictive algorithm may compare the heart activity as sensed by the microphone with a reference signature (e.g., a healthy heart activity, an abnormal heart activity, etc.) to classify the sensed heart activity based on similarity to the reference signature. Other predictive algorithms may be used.

In some examples, the method further includes capturing a second signal with a second sensor (e.g., a PPG, an electrode, an accelerometer, or a second microphone in a second ear of the user), processing the second signal to determine a second heart activity of the user, and detecting the heart pathology by applying the predictive algorithm to the heart activity of the user in the first microphone signal and the second heart activity of the user. The predictive algorithm may determine the heart pathology based on both the heart activity of the user and the second heart activity of the user.

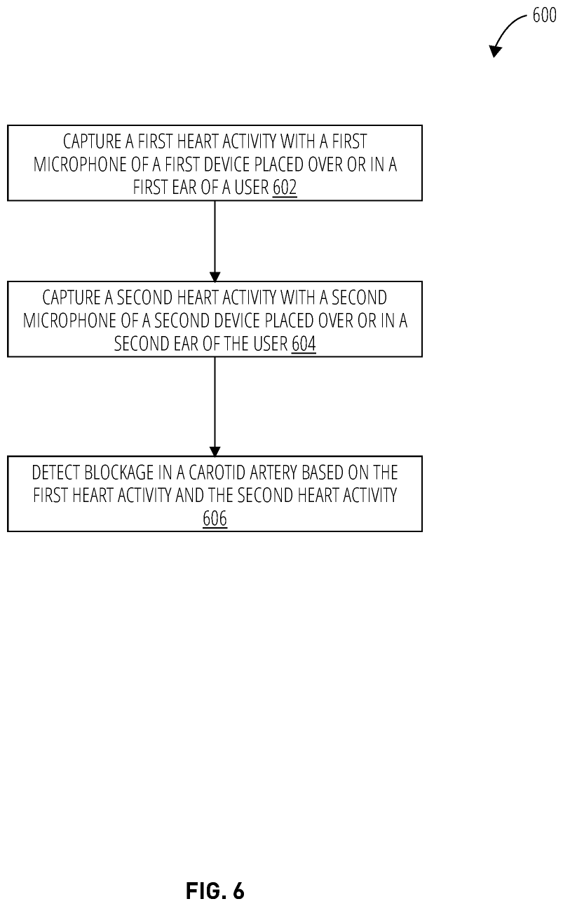

FIG. 6 illustrates an example of a method 600 for detecting a potential blockage of the carotid artery, based at least on heart activity sensed by a microphone, in accordance with some aspects. The method may be performed with various aspects described. The method may be performed by processing logic of an audio processing device.

At block 602, processing logic captures a first heart activity with a first microphone of a first device placed over or in a first ear of a user. At block 604, processing logic captures a second heart activity with a second microphone of a second device placed over or in a second ear of the user.

The first device and second device may include speakers used to output sound (e.g., a pilot signal) into each ear of the user, as described in other sections. At blocks 602 and 604, processing logic may process each of the microphone signals from the first microphone and the second microphone to extract or reconstruct the first heart activity and the second heart activity. In some examples, the first device and the second device may operate on a common clock. The first activity and the second activity, in such a case, are synchronized. In the case where they have separate clocks, a time synchronization technique may be utilized to synchronize the timing of the first heart activity and the second heart activity.

At block 606, processing logic detects blockage (or an indication of a blockage) in a carotid artery based on the first heart activity and the second heart activity. For example, processing logic may compare the first heart activity and the second heart activity to each other. If a difference in timing, strength, or both, between the first heart activity and the second heart activity is greater than a threshold, then processing logic may determine that the carotid artery has blockage. As such, the relationship (e.g., timing, strength, etc.) between the first heart activity and the second heart activity may indicate an elevated risk (e.g., a probability) that a blockage may be present. In some examples, processing logic may apply a machine learning model that is trained to predict the blockage based on the input waveforms of both the first heart activity and the second heart activity.

As described, processing logic may test the seal of each of the first device and the second device. Similarly, processing logic may validate that the user's activity or position does not taint the first heart activity or the second heart activity.

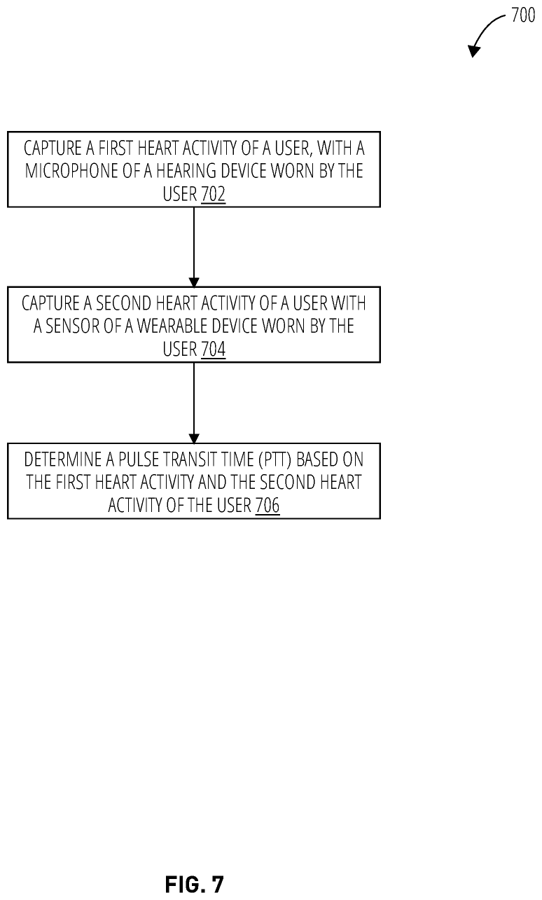

FIG. 7 illustrates an example of a method 700 for determining a pulse transit time (PTT), based at least on heart activity sensed by a microphone, in accordance with some aspects. The method may be performed with various aspects described. The method may be performed by processing logic of an audio processing device.

At block 702, processing logic captures a first heart activity of a user, with a microphone of a hearing device worn by the user. The hearing device may be an ear bud or a headphone that is worn in or on an ear of the user. As described in other sections, capturing the heart activity with a microphone on a hearing device may include sensing variations of a pilot signal as it reflects off the inside of a user's ear (e.g., the user's ear canal). A speaker of the hearing device may output the pilot signal.

At block 704, processing logic captures a second heart activity of a user with a sensor of a wearable device worn by the user. In some examples, the sensor may include an electrode that serves as an electrocardiogram (EKG). Additionally, or alternatively, the sensor may include a PPG sensor. The sensor may be worn over the user's chest, on a user's wrist, or at any other location.

At block 706, processing logic determines a pulse transit time (PTT) (or indication of the PTT) based on the first heart activity and the second heart activity of the user. The first heart activity and the second heart activity may be temporally synchronized to a common reference. The PTT may be determined based on or as the difference in timing between the first heart activity and second heart activity. In some examples, an indication of the PTT may be determined. For example, a relationship (e.g., a strength ratio, a delay, etc.) between the first heart activity and the second heart activity may be associated with the PTT. Processing logic may apply a predictive algorithm for both the first heart activity and the second heart activity to predict the PTT.

One aspect of the disclosure here is a method that captures a first heart activity with a first microphone of a first device placed over or in a first ear of a user; captures a second heart activity with a second microphone of a second device placed over or in a second ear of the user; and detects an indication of a blockage in a carotid artery based on the first heart activity and the second heart activity. Detecting the indication of the blockage may include detecting a difference in strength between the first heart activity and the second heart activity, and/or detecting a difference in timing between the first heart activity and the second heart activity. In one variation, detecting the indication of the blockage in the carotid artery includes using an artificial neural network to detect the blockage in the carotid artery based on both the first heart activity and the second heart activity.

The method may further include determining a seal of the first device to the ear of the user; and determining a reliability of the first heart activity of the user based on the seal. Or the method may further include determining a reliability of the first heart activity or the second heart activity of the user based on a position of the user.

An indication of the blockage in the carotid artery may be presented to a display.

In one version, the first device and the second device are respectively a first earbud and a second earbud of an earbud pair.

More generally, the first device and the second device may have separate housings.

In yet another aspect of the disclosure here, a method is performed in which a first heart activity of a user is captured with a first microphone of a hearing device worn by the user; and a second heart activity of a user is captured with a sensor of a wearable device worn by the user; and a pulse transit time (PTT) is determined based on the first heart activity and the second heart activity of the user. In one variation, determining the PTT includes detecting a difference in timing between the first heart activity and the second heart activity. In another variation, the sensor includes an electrode that electronically measures the second heart activity of the user. The method may further include synchronizing the first microphone and the sensor based on timestamps. The method may further include capturing a third heart activity of a user, with a second microphone, and determining the PTT further based on the second heart activity and the third heart activity, wherein the first microphone is worn in or on a first ear of the user, and the second microphone is worn in or on a second ear of the user. The PTT may be presented to a display. The method may further include determining a blood pressure of the user based at least on the PTT. In another variation, the method may further include determining a seal of the hearing device to an ear of the user and determining a reliability of the first heart activity of the user based on the seal. The method may further include determining a position of the user based on a second sensor and determining a reliability of the first heart activity of the user or the second heart activity of the user based on the position of the user.

In yet another aspect of the disclosure here, an audio processing device includes a processor configured to perform any of the methods described above.

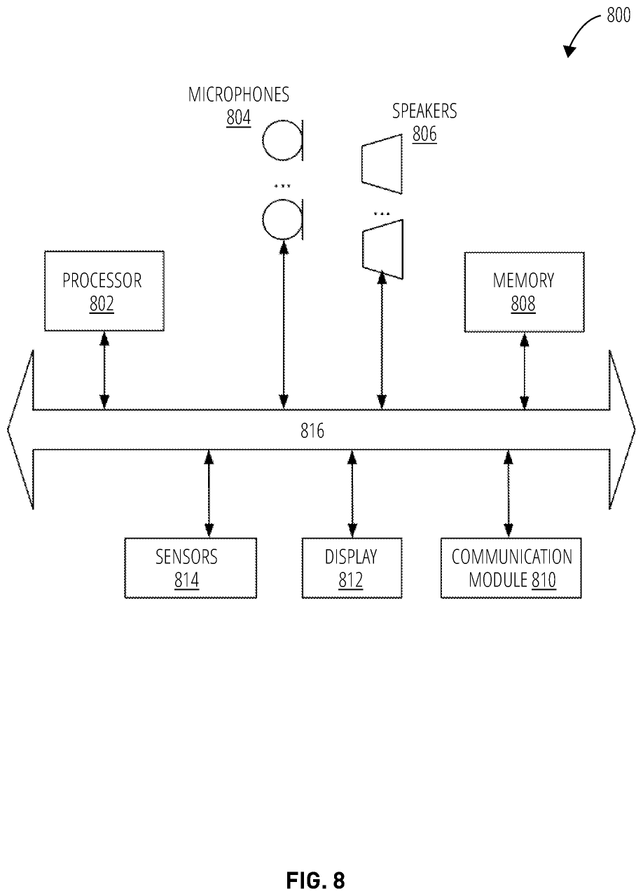

FIG. 8 illustrates an example of an audio processing system 800, in accordance with some aspects. The audio processing system can be a device such as, for example, a desktop computer, a tablet computer, a smart phone, a computer laptop, a smart speaker, a media player, a household appliance, a headphone set, a head mounted display (HMD), a watch, smart glasses, an infotainment system for an automobile or other vehicle, or another computing device. The system can be configured to perform the method and processes described in the present disclosure.

Although various components of an audio processing system are shown that may be incorporated into headphones, speaker systems, microphone arrays and entertainment systems, this illustration is merely one example of a particular implementation of the types of components that may be present in the audio processing system. This example is not intended to represent any particular architecture or manner of interconnecting the components as such details are not germane to the aspects herein. It will also be appreciated if other types of audio processing systems that have fewer or more components than shown can also be used. Accordingly, the processes described herein are not limited to use with the hardware and software shown.

The audio processing system can include one or more buses 816 that serve to interconnect the various components of the system. One or more processors 802 are coupled to bus as is known in the art. The processor(s) may be microprocessors or special purpose processors, system on chip (SOC), a central processing unit, a graphics processing unit, a processor created through an Application Specific Integrated Circuit (ASIC), or combinations thereof. Memory 808 can include Read Only Memory (ROM), volatile memory, and non-volatile memory, or combinations thereof, coupled to the bus using techniques known in the art. Sensors 814 can include an IMU and/or one or more cameras (e.g., RGB camera, RGBD camera, depth camera, etc.) or other sensors described herein. The audio processing system can further include a display 812 (e.g., an HMD, or touchscreen display).

Memory 808 can be connected to the bus and can include DRAM, a hard disk drive or a flash memory or a magnetic optical drive or magnetic memory or an optical drive or other types of memory systems that maintain data even after power is removed from the system. In one aspect, the processor 802 retrieves computer program instructions stored in a machine-readable storage medium (memory) and executes those instructions to perform operations described herein.

Audio hardware, although not shown, can be coupled to the one or more buses to receive audio signals to be processed and output by speakers 806. Audio hardware can include digital to analog and/or analog to digital converters. Audio hardware can also include audio amplifiers and filters. The audio hardware can also interface with microphones 804 (e.g., microphone arrays) to receive audio signals (whether analog or digital), digitize them when appropriate, and communicate the signals to the bus.

Communication module 810 can communicate with remote devices and networks through a wired or wireless interface. For example, communication modules can communicate over known technologies such as TCP/IP, Ethernet, Wi-Fi, 3G, 4G, 5G, Bluetooth, ZigBee, or other equivalent technologies. The communication module can include wired or wireless transmitters and receivers that can communicate (e.g., receive and transmit data) with networked devices such as servers (e.g., the cloud) and/or other devices such as remote speakers and remote microphones.

It will be appreciated that the aspects disclosed herein can utilize memory that is remote from the system, such as a network storage device which is coupled to the audio processing system through a network interface such as a modem or Ethernet interface. The buses can be connected to each other through various bridges, controllers and/or adapters as is well known in the art. In one aspect, one or more network device(s) can be coupled to the bus. The network device(s) can be wired network devices (e.g., Ethernet) or wireless network devices (e.g., Wi-Fi, Bluetooth). In some aspects, various aspects described (e.g., simulation, analysis, estimation, modeling, object detection, etc.,) can be performed by a networked server in communication with the capture device.

Various aspects described herein may be embodied, at least in part, in software. That is, the techniques may be carried out in an audio processing system in response to its processor executing a sequence of instructions contained in a storage medium, such as a non-transitory machine-readable storage medium (e.g., DRAM or flash memory). In various aspects, hardwired circuitry may be used in combination with software instructions to implement the techniques described herein. Thus, the techniques are not limited to any specific combination of hardware circuitry and software, or to any particular source for the instructions executed by the audio processing system.

In the description, certain terminology is used to describe features of various aspects. For example, in certain situations, the terms “module,” “processor,” “unit,” “renderer,” “system,” “device,” “filter,” “engine,” “block,” “detector”, “simulation”, “model”, and “component”, are representative of hardware and/or software configured to perform one or more processes or functions. For instance, examples of “hardware” include, but are not limited or restricted to an integrated circuit such as a processor (e.g., a digital signal processor, microprocessor, application specific integrated circuit, a micro-controller, etc.). Thus, different combinations of hardware and/or software can be implemented to perform the processes or functions described by the above terms, as understood by one skilled in the art. Of course, the hardware may be alternatively implemented as a finite state machine or even combinatorial logic. An example of “software” includes executable code in the form of an application, an applet, a routine or even a series of instructions. As mentioned above, the software may be stored in any type of machine-readable medium.

Some portions of the preceding detailed descriptions have been presented in terms of algorithms and symbolic representations of operations on data bits within a computer memory. These algorithmic descriptions and representations are the ways used by those skilled in the audio processing arts to convey the substance of their work most effectively to others skilled in the art. An algorithm is here, and, conceived to be a self-consistent sequence of operations leading to a desired result. The operations are those requiring physical manipulations of physical quantities. It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of an audio processing system, or similar electronic device, that manipulates and transforms data represented as physical (electronic) quantities within the system's registers and memories into other data similarly represented as physical quantities within the system memories or registers or other such information storage, transmission or display devices.

The processes and blocks described herein are not limited to the specific examples described and are not limited to the specific orders used as examples herein. Rather, any of the processing blocks may be re-ordered, combined, or removed, performed in parallel or in serial, as desired, to achieve the results set forth above. The processing blocks associated with implementing the audio processing system may be performed by one or more programmable processors executing one or more computer programs stored on a non-transitory computer readable storage medium to perform the functions of the system. All or part of the audio processing system may be implemented as, special purpose logic circuitry (e.g., an FPGA (field-programmable gate array) and/or an ASIC (application-specific integrated circuit)). All or part of the audio system may be implemented using electronic hardware circuitry that include electronic devices such as, for example, at least one of a processor, a memory, a programmable logic device or a logic gate. Further, processes can be implemented in any combination of hardware devices and software components.

In some aspects, this disclosure may include the language, for example, “at least one of [element A] and [element B].” This language may refer to one or more of the elements. For example, “at least one of A and B” may refer to “A,” “B,” or “A and B.” Specifically, “at least one of A and B” may refer to “at least one of A and at least one of B,” or “at least of either A or B.” In some aspects, this disclosure may include the language, for example, “[element A], [element B], and/or [element C].” This language may refer to either of the elements or any combination thereof. For instance, “A, B, and/or C” may refer to “A,” “B,” “C,” “A and B,” “A and C,” “B and C,” or “A, B, and C.”

While certain aspects have been described and shown in the accompanying drawings, it is to be understood that such aspects are merely illustrative of and not restrictive, and the disclosure is not limited to the specific constructions and arrangements shown and described, since various other modifications may occur to those of ordinary skill in the art.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

As described above, one aspect of the present technology is the gathering and use of data available from specific and legitimate sources. The present disclosure contemplates that in some instances, this gathered data may include personal information data that uniquely identifies or can be used to identify a specific person. Such personal information data can include demographic data, location-based data, online identifiers, telephone numbers, email addresses, home addresses, data or records relating to a user's health or level of fitness (e.g., vital signs measurements, medication information, exercise information), date of birth, or any other personal information.

The present disclosure recognizes that the use of such personal information data, in the present technology, can be used to the benefit of users. For instance, health and fitness data may be used, in accordance with the user's preferences to provide insights into their general wellness or may be used as positive feedback to individuals using technology to pursue wellness goals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are recognized as meeting or exceeding industry or governmental requirements for maintaining the privacy of users. Such information regarding the use of personal data should be prominent and easily accessible by users and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations that may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, users can select not to detect heart activity in a microphone signal of an ear-worn device. In addition to providing “opt in” and “opt out” options, the present disclosure contemplates providing notifications relating to the access or use of personal information.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users based on aggregated non-personal information data or a bare minimum amount of personal information, such as the content being handled only on the user's device or other non-personal information available to the content delivery services.

What is claimed is:

1. A method, comprising:
producing a microphone signal with a microphone of a device worn in or on an ear of a user;
processing the microphone signal to determine a first heart activity of the user; and
detecting an association between the first heart activity of the user and a heart pathology by applying a predictive algorithm based at least on the first heart activity.

2. The method of claim 1, further comprising:
capturing a second signal with a second sensor;
processing the second signal to determine a second heart activity of the user; and
detecting an association between the second heart activity or the first heart activity and the heart pathology by applying the predictive algorithm to both the first heart activity of the user and the second heart activity of the user.

3. The method of claim 2, wherein the second sensor includes an accelerometer.

4. The method of claim 2, wherein the second sensor includes an electrode.

5. The method of claim 2, wherein the second sensor includes a second microphone worn in a second ear of the user.

6. The method of claim 2, wherein the second sensor includes a PPG.

7. The method of claim 1, wherein the predictive algorithm includes a neural network that is trained to detect the association between the first heart activity of the user and the heart pathology, based on the first heart activity of the user.

8. The method of claim 1, wherein the predictive algorithm detects the association between the first heart activity of the user and the heart pathology, based on similarity between the first heart activity and one or more reference signatures of the heart pathology.

9. The method of claim 1, wherein processing the microphone signal includes applying a low pass filter to the microphone signal.

10. The method of claim 1, wherein processing the microphone signal includes sensing an ultrasound signal in the microphone signal.

11. The method of claim 1, wherein processing the microphone signal sensing an infrasound signal in the microphone signal.

12. The method of claim 1, wherein the heart pathology includes bradycardia or tachycardia.

13. The method of claim 1, wherein the first heart activity includes a heartbeat or a heart movement.

14. The method of claim 1, further comprising determining a seal of the device to the ear of the user to determine a reliability of the first heart activity of the user.

15. The method of claim 1, further comprising determining a position of the user based on a second sensor and determining a reliability of the first heart activity of the user based on the position of the user.

16. The method of claim 1, further comprising enhancing a signal of the first heart activity using an active noise cancellation and one or more machine learning processes.

17. The method of claim 1, further comprising presenting an indication of the heart pathology to a display.

18. The method of claim 1, further comprising processing the microphone signal to detect respiratory activity and detecting the association between the first heart activity and the heart pathology further based on the respiratory activity.

19. A method, comprising:
capturing a first heart activity with a first microphone of a first device placed over or in a first ear of a user;
capturing a second heart activity with a second microphone of a second device placed over or in a second ear of the user; and
detecting indication of a blockage in a carotid artery based on the first heart activity and the second heart activity.

20. The method of claim 19, wherein detecting the indication of the blockage includes detecting a difference in strength between the first heart activity and the second heart activity.
